(19) United States
(12) Patent Application Publication
(10) Pub. No.: US 2011/0099694 A1
(43) Pub. Date: May 5, 2011

(54) Apparatus for Mitigating a Loss of or Damage to Protective Eye Goggles used in Conjunction with a Sporting Helmet

(76) Inventor: James Paul Guida, Miller Place, NY (US)

(21) Appl. No.: 12/610,990

(22) Filed: Nov. 2, 2009

(51) Int. Cl. A42B 1/24; A61F 9/00

(52) U.S. Cl. 2/422; 2/425; 2/10

(57) ABSTRACT

An apparatus for mitigating a loss of or damage to protective eye goggles used in conjunction with a sporting helmet includes two goggle strap clips joinable to the sporting helmet on opposing sides. Each of the goggle strap clips includes a long, flat bottom surface for joining to the sporting helmet. Two upper legs are L-shaped and have an opening between ends of the upper legs for enabling a goggle strap to pass through the goggle strap clip and be secured in place. The ends are curved for enabling easy installation and removal the goggle strap from the goggle strap clip. A seamless construction and smooth edges mitigates snags in the goggle strap as it is installed or removed. Smooth outer faces enables logos to be embossed or printed thereon. A flexible material for enables the flat bottom surface to conform to a curved shape of the sporting helmet.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to sports equipment. More particularly, the invention relates to a goggle strap clip that generally prevents goggles from slipping off a helmet when the goggles are raised from the normal use position.

### BACKGROUND OF THE INVENTION

Most skiers today wear a protective helmet, and goggles are the most popular form of eye protection worn in conjunction with the helmet. When the goggles are in place while skiing, they remain in a secure position with the helmet. However, problems arise once the skier enters the lift line or stops for a break, and decides to lift his goggles up while still wearing his helmet. Once the goggles are lifted, the tension in the goggle strap causes the goggles to be pulled back off the top of the helmet. This either causes the goggles to fly off the backside of the helmet or leaves the goggles hanging from a rear-mounted strap on the helmet if the helmet is so equipped. On many occasions this happens while riding a chair lift and results in a person loosing a very expensive pair of goggles.

FIG. 1 illustrates an exemplary helmet 100 with a rear mounted helmet strap 103, in accordance with the prior art. Helmet manufacturers have provided helmet strap 103 at the rear of helmet 100 through which a goggle strap 105 from a pair of goggles 107 passes in an attempt to catch goggles 107 if they slide off the rear of helmet 100 when raised from the engaged position. Helmet strap 103 generally eliminates the chance of loosing goggles 107. However, the damage caused by goggles 107 swinging from rear goggle strap 105 can cause scratches to the delicate goggle lenses, specifically when the lenses come into contact with the steel tubing of a chair lift. Other than a rear mounted helmet strap, there is nothing in the market place which handles the specific problem of keeping goggles secure in both an engaged position and a disengaged position.

In view of the foregoing, there is a need for improved techniques for providing means for safely securing goggles onto a helmet in both the engaged position and the disengaged position.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary helmet with a rear goggle strap, in accordance with the prior art;

FIGS. 2A, 2B and 2C illustrate an exemplary goggle strap clip, in accordance with an embodiment of the present invention. FIG. 2A is a cross sectional view of the goggle strap clip. FIG. 2B is a front elevation view of the goggle strap clip, and FIG. 2C is a perspective view of the goggle strap clip;

FIGS. 3A and 3B illustrate an exemplary goggle strap clip in use on a helmet with goggles, in accordance with an embodiment of the present invention. FIG. 3A shows the goggles in an engaged position on the helmet, and FIG. 3B shows the goggles in a disengaged position on the helmet.

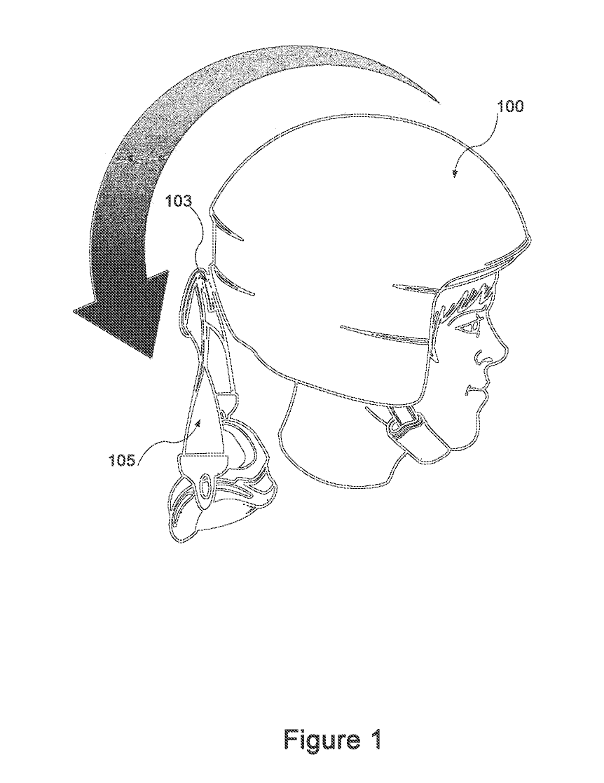

Unless otherwise indicated illustrations in the figures are not necessarily drawn to scale.

### SUMMARY OF THE INVENTION

To achieve the forgoing and other objects and in accordance with the purpose of the invention, an apparatus for mitigating a loss of or damage to protective eye goggles used in conjunction with a sporting helmet is presented.

In one embodiment an apparatus for mitigating a loss of or damage to protective eye goggles used in conjunction with a sporting helmet is presented. The apparatus includes two goggle strap clips joinable to the sporting helmet on opposing sides, each of the goggle strap clips including means for joining to the sporting helmet, means for enabling a goggle strap to pass through the goggle strap clip and be secured in place and means for enabling easy installation and removal the goggle strap from the goggle strap clip.

In another embodiment an apparatus for mitigating a loss of or damage to protective eye goggles used in conjunction with a sporting helmet is presented. The apparatus includes two goggle strap clips joinable to the sporting helmet on opposing sides, each of the goggle strap clips includes a long, flat bottom surface for joining to the sporting helmet, and two upper legs being L-shaped and having an opening between ends of the upper legs for enabling a goggle strap to pass through the goggle strap clip and be secured in place, the ends being curved for enabling easy installation and removal the goggle strap from the goggle strap clip.

In another embodiment an apparatus for mitigating a loss of or damage to protective eye goggles used in conjunction with a sporting helmet is presented. The apparatus includes two goggle strap clips joinable to the sporting helmet on opposing sides, each of the goggle strap clips includes a long, flat bottom surface for joining to the sporting helmet. Two upper legs are L-shaped and have an opening between ends of the upper legs for enabling a goggle strap to pass through the goggle strap clip and be secured in place. The ends are curved for enabling easy installation and removal the goggle strap from the goggle strap clip. A seamless construction and smooth edges mitigates snags in the goggle strap as it is installed or removed from the goggle strap clip. A coloring matches the sporting helmet or the eye goggles. Smooth outer faces enable logos to be embossed or printed thereon. A flexible material enables the flat bottom surface to conform to a curved shape of the sporting helmet.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

Detailed descriptions of the preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

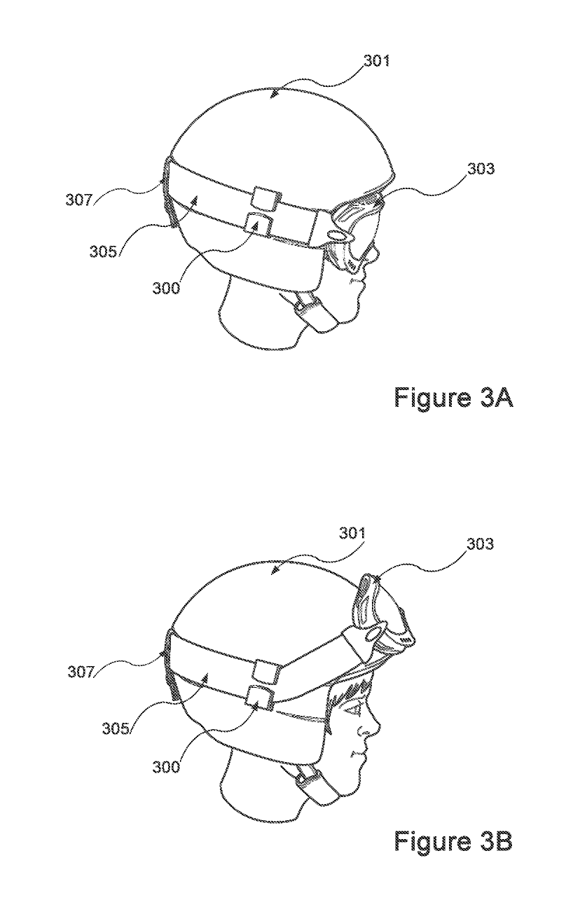

Preferred embodiments of the present invention generally prevent the loss of or damage to protective eye goggles that are used in conjunction with a sporting helmet such as, but not limited to, a ski helmet. Preferred embodiments can be adapted to a user's current gear to correct the problem of the goggles slipping off of the helmet with out the need to purchase all new equipment. When a strap clip according to a preferred embodiment is installed on an existing helmet and the goggle strap is passed through the clip, the goggles can no longer slip off the back of the helmet if the user raises the goggles from the engaged position. Although a ski helmet and goggles are used for illustration purposes in the following description and in FIGS. 3A and 3B, preferred embodiments of the present invention may be designed and built to adapt to any type of helmet such as, but not limited to, motorcycle helmets, helmets for use with all terrain vehicles (ATVs), military and law enforcement helmets, etc. Preferred embodiments of the present invention may be used in varied commercial fields and by a wide variety of users such as, but not limited to, snowboarders, skiers, motocross racers, etc. Preferred embodiments can adapt to varied situations in which a user wears protective eye goggles in conjunction with a protective helmet. A desired characteristic of preferred embodiments of the present invention is to end the constant frustration for people who lose or damage expensive protective goggles simply from having the goggles fly off the back of their helmet. The popularity of skiing and snowboarding alone creates a significant need for a solution to this problem.

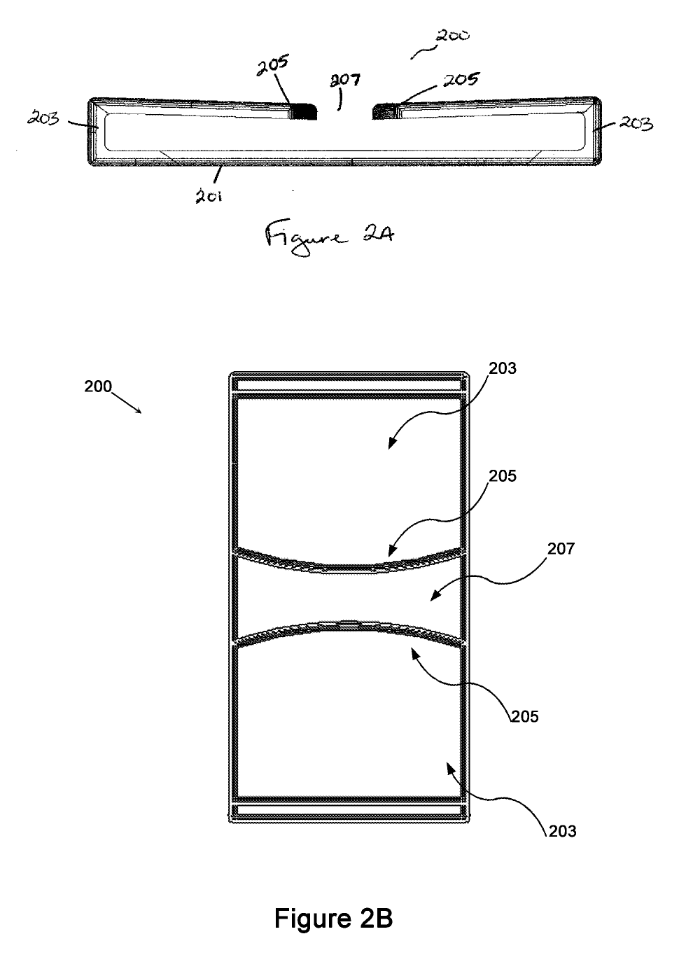

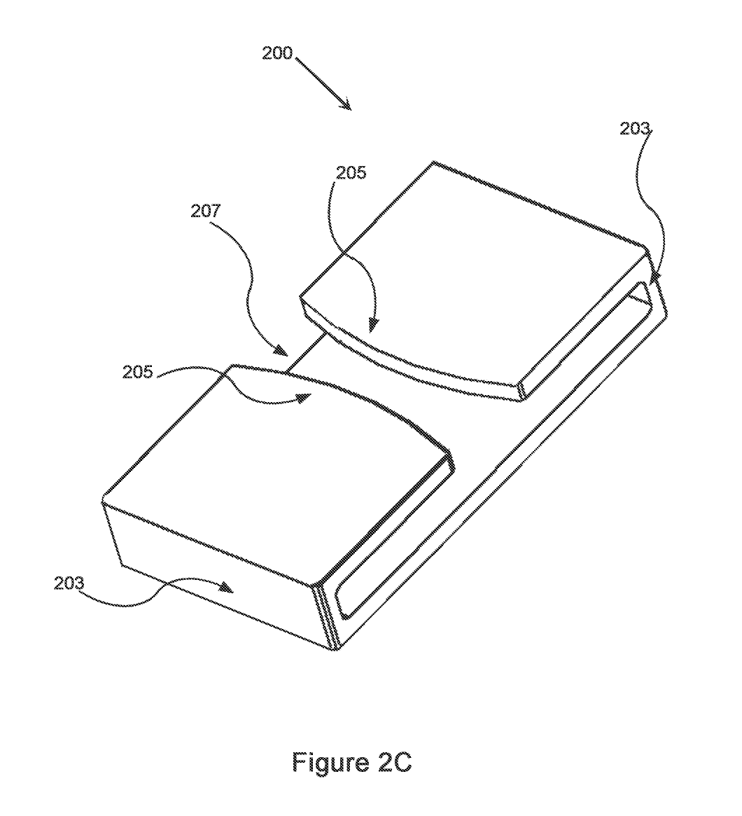

FIGS. 2A, 2B and 2C illustrate an exemplary goggle strap clip 200, in accordance with an embodiment of the present invention. FIG. 2A is a cross sectional view of goggle strap clip 200. FIG. 2B is a front elevation view of goggle strap clip 200, and FIG. 2C is a perspective view of goggle strap clip 200. In the present embodiment, goggle strap clip 200 comprises a long, flat bottom surface 201 and two upper legs 203. Bottom surface 201 attaches to a helmet in typical use of the present embodiment. Upper legs 203 are L-shaped to enable a goggle strap to pass through goggle strap clip 200 and be secured in place. In the present embodiment, ends 205 of upper legs 203 at an opening 207, in which the goggle strap is inserted into goggle strap clip 200, are curved. The nature of the curve is to make it easy to install and remove the goggle straps from goggle strap clip 200. However, alternate embodiments may be implemented with non-curved arms.

In the present embodiment, goggle strap clip 200 is injection molded in 1/16″ thick polypropylene material and measures about 1¾″ high and 1″ wide. However, alternate embodiments may be constructed using various different means such as, but not limited to, thermoforming and extrusion and may be made of various different materials such as, but not limited to, different type of plastics, metal, rubber, etc. Alternate embodiments may also be made in different sizes. For example, without limitation, a smaller goggle strap clip may be made for use with children's helmets. In the present embodiment, goggle strap clip 200 is preferably made from a flexible material to enable flat bottom surface 201 to conform to the curved shape of a helmet; however, rigid goggle strap clips that are shaped to conform to specific styles of helmets may be implemented in alternate embodiments. The lightweight construction of goggle strap clip 200 enables goggle strap clip 200 to be installed on a helmet with very high bond (VHB) tape; however, other installation means may be used such as, but not limited to, adhesives, hook and loop material, bolts or screws, snaps, etc. In some embodiments goggle strap clips may be built into the sides of a helmet.

In the present embodiment, goggle strap clip 200 is attached to a helmet in such a fashion that once the straps of the protective eye goggles pass through goggle strap clip 200, goggle strap clip 200 generally prevents the goggles from sliding back off the helmet if the user raises the goggles from the engaged position, as shown by way of example in FIG. 3B. Referring to FIG. 2C, goggle strap clip 200 preferably has smooth edges and a seamless construction that reduces snags in the goggle strap as it is placed in goggle strap clip 200. Goggle strap clip 200 may be made in various different colors to match helmets or goggles of different colors. Furthermore, goggle strap clip 200 comprises smooth outer faces on which logos may be embossed or printed.

FIGS. 3A and 3B illustrate an exemplary goggle strap clip 300 in use on a helmet 301 with goggles 303, in accordance with an embodiment of the present invention. FIG. 3A shows goggles 303 in an engaged position on helmet 301, and FIG. 3B shows goggles 303 in a disengaged position on helmet 301. When goggle strap clips 300 are mounted on both sides of helmet 301 in line with where a goggle strap 305 falls when in the engaged position, goggle strap clips 300 hold strap 305 in place at each side of helmet 301. Goggle strap clip 300 is shown in use with a rear helmet strap 307; however, goggle strap clips 300 may also be used without a rear helmet strap. Securing goggle strap 305 at the sides generally prevents goggles 303 from snapping backwards once the user raises them from the engaged position to the disengaged position. Referring to FIG. 3B, the location of goggle strap clips 300 enables the sides of strap 305 to remain in place when goggles 303 are moved up into the disengaged position. This generally prevents goggles 303 from sliding back any further on helmet 301 or flying completely off of helmet 301 and insures the safety of goggles 303 as well as reducing the likelihood of unnecessary scratching or loss.

Having fully described at least one embodiment of the present invention, other equivalent or alternative methods of providing a goggle strap clip according to the present invention will be apparent to those skilled in the art. The invention has been described above by way of illustration, and the specific embodiments disclosed are not intended to limit the invention to the particular forms disclosed. For example, the particular implementation of the goggle strap clip may vary depending upon the particular shape of the bottom surface. The bottom surfaces described in the foregoing were directed to rectangular implementations; however, similar techniques are to provide bottom surface and therefore goggle strap clips in various different shapes such as, but not limited to, squares, circles, ovals, triangles, or decorative shapes such as, but not limited to, hearts, stars, flowers, etc. Non-rectangular implementations of the present invention are contemplated as within the scope of the present invention. The invention is thus to cover all modifications, equivalents, and alternatives falling within the spirit and scope of the following claims.

Claim elements and steps herein have been numbered and/or lettered solely as an aid in readability and understanding. As such, the numbering and lettering in itself is not intended to and should not be taken to indicate the ordering of elements and/or steps in the claims.

What is claimed is:

1. An apparatus for mitigating a loss of or damage to protective eye goggles used in conjunction with a sporting helmet, the apparatus comprising:
two goggle strap clips joinable to the sporting helmet on opposing sides, each of said goggle strap clips comprising means for joining to the sporting helmet, means for enabling a goggle strap to pass through said goggle strap clip and be secured in place and means for enabling easy installation and removal the goggle strap from said goggle strap clip.

2. The apparatus as recited in claim 1, further comprising means for mitigating snags in the goggle strap as it is installed or removed from said goggle strap clip.

3. The apparatus as recited in claim 1, further comprising means for matching the sporting helmet or eye goggles.

4. The apparatus as recited in claim 1, further comprising means for enabling logos to be embossed or printed thereon.

5. The apparatus as recited in claim 1, further comprising means for enabling said joining means to conform to a curved shape of the sporting helmet.

6. An apparatus for mitigating a loss of or damage to protective eye goggles used in conjunction with a sporting helmet, the apparatus comprising:
two goggle strap clips joinable to the sporting helmet on opposing sides, each of said goggle strap clips comprising a long, flat bottom surface for joining to the sporting helmet, and two upper legs being L-shaped and having an opening between ends of said upper legs for enabling a goggle strap to pass through said goggle strap clip and be secured in place, said ends being curved for enabling easy installation and removal the goggle strap from said goggle strap clip.

7. The apparatus as recited in claim 6, further comprising a seamless construction and smooth edges for mitigating snags in the goggle strap as it is installed or removed from said goggle strap clip.

8. The apparatus as recited in claim 6, further comprising a coloring for matching the sporting helmet or the eye goggles.

9. The apparatus as recited in claim 6, further comprising smooth outer faces for enabling logos to be embossed or printed thereon.

10. The apparatus as recited in claim 6, further comprising a flexible material for enabling said flat bottom surface to conform to a curved shape of the sporting helmet.

11. The apparatus as recited in claim 10, wherein said flexible material comprises polypropylene material for a lightweight construction.

12. The apparatus as recited in claim 6, wherein said goggle strap clips are joined to the sporting helmet with a very high bond (VHB) tape.

13. The apparatus as recited in claim 6, wherein said goggle strap clips are built into the sides of the sporting helmet.

14. The apparatus as recited in claim 6, wherein the sporting helmet has a rear helmet strap and the apparatus is used in conjunction with the rear helmet strap.

15. An apparatus for mitigating a loss of or damage to protective eye goggles used in conjunction with a sporting helmet, the apparatus comprising:
two goggle strap clips joinable to the sporting helmet on opposing sides, each of said goggle strap clips comprising a long, flat bottom surface for joining to the sporting helmet, two upper legs being L-shaped and having an opening between ends of said upper legs for enabling a goggle strap to pass through said goggle strap clip and be secured in place, said ends being curved for enabling easy installation and removal the goggle strap from said goggle strap clip, a seamless construction and smooth edges for mitigating snags in the goggle strap as it is installed or removed from said goggle strap clip, a coloring for matching the sporting helmet or the eye goggles, smooth outer faces for enabling logos to be embossed or printed thereon and a flexible material for enabling said flat bottom surface to conform to a curved shape of the sporting helmet.

16. The apparatus as recited in claim 15, wherein said flexible material comprises polypropylene material for a lightweight construction.

17. The apparatus as recited in claim 15, wherein said goggle strap clips are joined to the sporting helmet with a very high bond (VHB) tape.

18. The apparatus as recited in claim 15, wherein said goggle strap clips are built into the sides of the sporting helmet.

19. The apparatus as recited in claim 15, wherein the sporting helmet has a rear helmet strap and the apparatus is used in conjunction with the rear helmet strap.
